(19) United States
(12) Patent Application Publication
(10) Pub. No.: US 2006/0148897 A1
(43) Pub. Date: Jul. 6, 2006

(54) Method for sterilization using in situ gelling materials

(76) Inventors: Brent Vernon, Mesa, AZ (US); Kelly H Roy, Phoenix, AZ (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/527,543

(22) PCT Filed: Sep. 15, 2003
(86) PCT No.: PCT/US03/28927

§ 371 Date: Jan. 18, 2006

Related U.S. Application Data
(60) Provisional application No. 60/410,724, filed on Sep. 13, 2002.

(51) Int. Cl. A61K 31/225

(52) U.S. Cl. 514/547

(57) ABSTRACT

A method for sterilizing a female or male patient is provided. The method comprises introducing into a reproductive duct of the patient a composition comprising a nucleophilic component, such as a thiol, and a component containing a conjugated unsaturated bond, such as an acrylate. The composition crosslinks within the reproductive duct. Within a female patient, the composition is introduced into one or both uterine tubes. The composition gels in the uterine tubes, thereby blocking the uterine tubes and preventing conception.

### BACKGROUND

A popular method for permanent contraception is tubal ligation, a surgical procedure where the fallopian tubes are cut and often removed, cauterized or sutured closed. As with any surgery, there are medical risks associated with this kind of procedure. In addition, the financial cost is high, making it economically unavailable to a significant part of the population. Another disadvantage is the permanent damage done to the fallopian tube.

An alternative (less invasive) solution has been the use of intrauterine devices. These can be highly effective for long-term contraception, with a low failure rate of 0.2-0.5% reported over ten years worldwide. However, there are a number of potential risks associated with the traditional types of intrauterine devices available on the market today. Some of the more serious risks include pelvic inflammatory disease, ectopic pregnancy, perforation of the wall of the uterus by the device, expulsion of the device, as well as permanent infertility. An additional potential side effect of copper IUDs is an increase in bleeding and cramping during the menstrual cycle, particularly during the first year of insertion.

Due to the complications and disadvantages associated with tubal ligation and intrauterine devices, new methods for sterilization, and more particularly reversible sterilization, are desirable.

### SUMMARY OF THE INVENTION

The present invention is directed to improved methods for sterilization that utilize in situ gelling materials. In one embodiment, the invention is directed to a method for sterilizing a patient comprising introducing into a reproductive duct of the patient a composition, preferably a fluid composition, comprising a nucleophilic component and a component containing a conjugated unsaturated bond, whereby the composition crosslinks within the reproductive duct.

In a more particular embodiment, the invention is directed to a method for sterilizing a female patient comprising introducing into a uterine tube of the patient a composition, preferably a fluid composition, comprising a nucleophilic component and a component containing a conjugated unsaturated bond, whereby the composition crosslinks within the uterine tube. For the average female patient, who has two functioning uterine tubes, the composition is preferably introduced into both uterine tubes. To the extent that the patient has only one functioning uterine tube, the composition need only be introduced into the one functioning uterine tube to prohibit conception.

In another more particular embodiment, the invention is directed to a method for sterilizing a male patient comprising introducing into at least one ejaculatory duct and/or the vas deferens of the patient a composition, preferably a fluid composition, comprising a nucleophilic component and a component containing a conjugated unsaturated bond, whereby the composition crosslinks within the at least one ejaculatory duct and/or the vas deferens. Again, for the average male patient, who has two functioning ejaculatory ducts, the composition is preferably introduced into both ejaculatory ducts. To the extent that the patient has only one functioning ejaculatory duct, the composition need only be introduced into the one functioning ejaculatory duct.

Injectable, in situ gelling biomaterials are attractive for use in sterilization because of increased ease of use and reduced invasiveness associated with their application as implanted materials. Further, the use of the Michael-type addition reaction, being pH dependent, can permit premixing of the reagents without reaction, and the reaction can be accelerated at a desired time by addition of a base.

### DETAILED DESCRIPTION

The invention is directed to methods for sterilizing female as well as male patients. Generally, the methods comprise introducing into a reproductive duct of the patient a liquid mixture of a nucleophilic component and a component containing a conjugated unsaturated bond, also referred to herein as a conjugated unsaturated compound. The mixture gels within the reproductive duct to form a gelled composition that blocks the reproductive duct. If desired, the gelled composition can be subsequently removed to thereby reverse the sterilization procedure. In female patients, the liquid mixture is introduced into one, and preferably both, of the uterine tubes, which will depend on whether both uterine tubes are present and functioning. In male patients, the liquid mixture is preferably introduced into the ejaculatory tract and/or the vas deferens.

The invention is based on a chemical reaction in which two or more precursor components, namely a nucleophilic component and a component containing a conjugated unsaturated bond, are polyermized or crosslinked in situ in a self-selective manner. These two precursor components are self-selective in their reaction rates. In other words, the nucleophilic component reacts faster with the component containing a conjugated unsaturated bond than with other components present during the reaction, and the component containing a conjugated unsaturated bond reacts faster with the nucleophilic compound than with other components present during the reaction.

The functionalities of the precursor components will affect the resulting polymerization product. The word “functionality” as used herein refers to the number of reactive sites, as generally used in polymer science. Mixing two components each having a functionality of two results in a linear polymeric biomaterial. If one of the components has a funtionality of more than two, mixing of the components will result in a cross-linked polymeric biomaterial. In cross-linked biomaterials, the components can be very hydrophilic, and the overall material can yet remain as an intact solid, not dispersing throughout the body. If such a non-dispersing system is desired for a linear polymeric biomaterial, it is useful if at least one precursor component be hydrophobic, such that the resulting biomaterial also be insoluble in water or body fluids.

The present invention makes use of a Michael-type addition reaction between the nucleophilic component and the component containing a conjugated unsaturated bond. The reaction can be exemplified as follows:

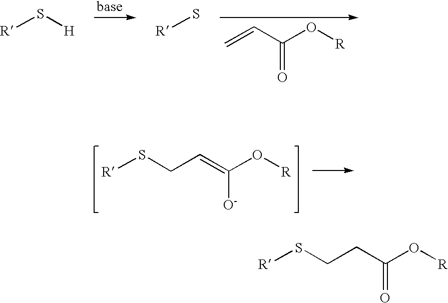

Such Michael-type addition reactions can be performed on a wide variety of conjugated unsaturated compounds in accordance with the invention. Exemplary conjugated unsaturated compounds include those having structures 1 to 20 set forth below. In these structures, P indicates an oligomeric or polymeric structure, examples of which are discussed further below. In structures 1 to 20, P is intended as terminated with a CH₂, CH or C group. Reactive double bonds can be conjugated to one or more carbonyl groups in a linear ketone, ester or amide structure (1, 2) or to two in a ring system, as in a maleic or paraquinoid derivative (3, 4, 5, 6, 7, 8, 9, 10). In the latter case, the ring can be fused to give a naphthoquinone (6, 7, 10) or a 4,7-benzimidazoledione (8), and the carbonyl groups can be converted to an oxime (9, 10). The double bond can be conjugated to a heteroatom-heteroatom double bond, such as a sulfone (11), a sulfoxide (12), a sulfonate or a sulfonamide (13), or a phosphonate or phosphonamide (14). Alternatively, the double bond can be conjugated to an electron-poor aromatic system, such as a 4-vinylpirydinium ion (15). Triple bonds can be used in conjugation with carbonyl or heteroatom-based multiple bonds (16, 17, 18, 19, 20).

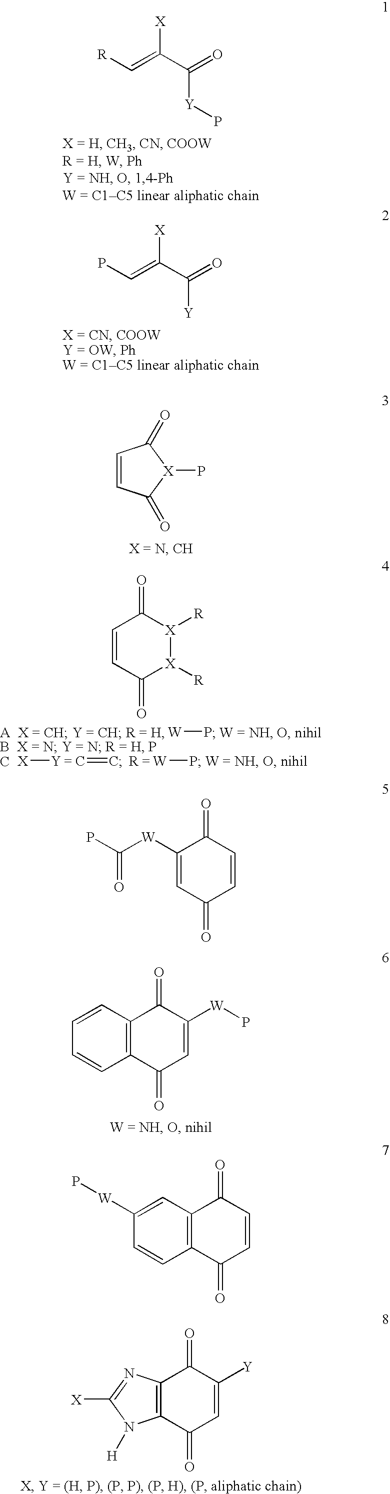

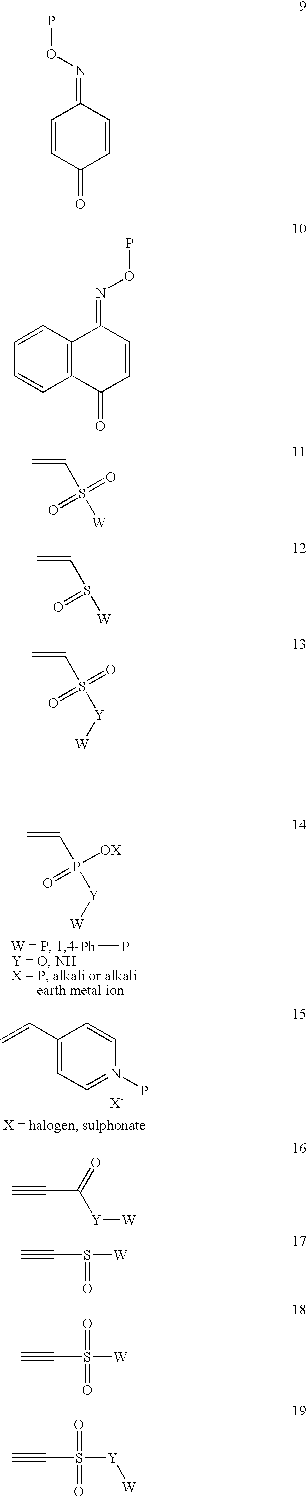

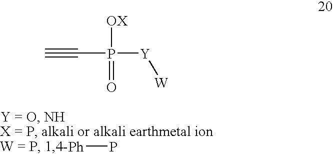

Structures such as 1 and 2 are based on the conjugation of a carbon-carbon double bond with one or two electron-withdrawing groups. One of them is always a carbonyl, increasing the reactivity passing from an amide, to an ester, and then to a phenone structure. The nucleophilic addition is easier upon decreasing the steric hindrance, or increasing the electron-withdrawing power in the alpha-position: CH₃<H<COOW<CN.

The higher reactivity obtained by using the last two structures can be modulated by varying the bulkiness of the substituents in the beta-position. where the nucleophilic attack takes place. The reactivity decreases in the order P<W<Ph<H. So the position of P too can be used to tune the reactivity towards nucleophiles. This family includes some 10 compounds for which a great deal is known about their toxicology and use in medicine. For example, water-soluble polymers with acrylates and methacrylates on their termini are polymerized (by free radical mechanisms) in vivo, in hydrogel sealants and bone cements, respectively. Thus. acrylate and methacrylate-containing polymers have been seen in the body before in clinical products, but for use with a dramatically different chemical reaction scheme.

The structures 3 to 10 exhibit very high reactivity towards nucleophiles, due both to the cis configuration of the double bond and the presence of two electron-withdrawing groups. Unsaturated ketones react faster than amides or imides, due to the stronger electronegativity of these carbonyl groups. So, cyclopentendione derivatives react faster than maleimidic ones (3), and para-quinones react faster than maleic hydrazides (4) and also faster than cyclohexanones, due to more extended conjugation. The highest reactivity is shown by naphthoquinones (7).

P can be placed in positions where it does not reduce the reactivity of the unsaturated group, that is in the opposite part of the ring (3, 5), on another ring (7, 8) or O-linked through a para-quinone mono-oxime (9, 10). P can be also linked to the reactive double bond (6, 8), particularly if the nucleophilic addition rate is to be decreased.

The activation of double bonds to nucleophilic addition can be obtained also by using hetheroatom-based electron-withdrawing groups. In fact, heteroatom-containing analogs of ketones (11, 12), esters and amides (13, 14) provide a similar electronic behavior. Structures 13 and 14 can also be used as easily hydrolyzable groups that can promote a quick gel degradation. The reactivity towards nucleophilic addition increases with electronegativity of the group, that is in the order 11>12>13>14, and is enhanced by the linkage with an aromatic ring. A strong activation of double bonds can also be obtained, using electron-withdrawing groups based on aromatic rings. Any aromatic structure containing a pyridinium-like cation (e.g., derivatives of quinoline, imidazole, pyrazine, pyrimidine, pyridazine, and similar sp containing compounds) strongly polarizes the double bond and makes possible quick Michael-type additions.

Carbon-carbon triple bonds, conjugated with carbon- or heteroatom-based electron-withdrawing groups, can easily react with sulphur nucleophiles, to give products from simple and double addition. The reactivity is influenced by the substituents, as for the double bond-containing analogous compounds.

Particularly preferred conjugated unsaturated compounds for use in the invention include acrylates, vinylsulfones, acrylamides, quinones and vinylpyridiniums, with acrylates being particularly preferred.

The nucleophiles that are useful are those that are reactive towards conjugated unsaturated groups by way of Michael-type addition reactions. The reactivity of the nucleophile depends on the identity of the unsaturated group, but the identity of the unsaturated group is first limited by its reaction with water at physiologic pH. Thus, useful nucleophiles will generally be more nucleophilic than water at physiologic pH. Preferred nucleophiles are those that are commonly found in biological systems. for reasons of toxicology, but ones that are not commonly found free in biological systems outside of cells. Thus, while there may be examples in which amines can be employed as effective nucleophiles, the most preferred nucleophile is the thiol. Thiols are present in biological systems outside of cells in paired form, as disulfide linkages. When the highest degree of self-selectivity is desired (e.g., when a therapeutic protein is incorporated, when the gelation reaction is conducted in the presence of tissue and chemical modification of that tissue is not desirable), then a thiol will represent the strong nucleophile of choice.

There are other situations, however, when the highest level of self-selectivity may not be necessary. This would include situations when no therapeutic protein is incorporated and when the gelation reaction is conducted in situ, but when chemical bonding to the tissue is either desirable or is not undesirable. In these cases, an amine may serve as an adequate nucleophile. Here, particular attention is paid to the pH, in that the deprotonated amine is a much stronger nucleophile than the protonated amine. Thus, for example, the alpha amine on a typical amino acid (pK as low as 8.8 for asparagine, average of 9.0 for all 20 common amino acids except proline) has a much lower pK than the side chain epsilon amine of lysine (pK 10.80). As such, if particular attention is paid to the pK of an amine used as the strong nucleophile, substantial self-selectivity can be obtained. Proteins have only one alpha amine (on the N-terminus). By selection of an amine with a low pK, and then formulation of the final precursor solution such that the pH were near that pK, one could favor reaction of the unsaturation provided with the amine provided, rather than other amines present in the system. In cases where no self selectivity is desired, one need pay less attention to the pK of the amine used as the nucleophile. However to obtain reaction rates that are acceptably fast one must adjust the pH of the final precursor solution such that an adequate number of these amines are deprotonated.

The term “nucleophilic group” as used herein includes not only the functional groups themselves (e.g., thiol or amine), but also molecules that contain the functional group (e.g., cysteine or cystyl residue, or lysine or lysyl residue). The nucleophilic groups may be contained in molecules with great flexibility in overall structure. For example, a difunctional nucleophile could be presented in the form of Nuc-P-Nuc, where P has the meaning discussed above, and Nuc refers to the nucleophile. Likewise, a branched polymer P could be derivatized with a number of nucleophiles to create P-(Nuc)i, where i=2 would be useful. Nuc needs not be displayed at the chain termini of P. For example, a repeating structure could be envisioned: (P Nuc)ᵢ where i=2 would be useful. Clearly, not all of the P or Nuc groups in such a structure need to be identical. It is only necessary that one nucleophilic precursor contain greater than or equal to two such Nuc groups.

Likewise, similar structures of P and the conjugated unsaturated groups described above may be formed. It is only necessai that one conjugated unsaturated precursor contain greater than or equal to two such conjugated unsaturated groups.

It should be noted and understood, that it is not necessary that both precursor components, for example, both the nucleophilic precursor component and the conjugated unsaturated precursor component, actually be polymeric in the usual sense of the word. It is only the functionality that matters. In practice, it is convenient if at least one component is polymeric in the usual sense of the word, but this is not absolutely necessary. For example, useful. materials result from the reaction of a PEG triacrylate with dicysteine, and likewise, useful materials result from the reaction of a PEG trithiol and a low molecular weight diacrylate. Further, useful materials for some applications also result from reaction of a dicysteine and a low molecular diacrylate.

In practice, it is convenient and useful when one or more precursor component is polymeric in the usual sense of the word. In these cases, P can be a synthetic hydrophilic polymer, a synthetic hydrophobic polymeric liquid, a synthetic hydrophobic polymer that is soluble in solvents of acceptable toxicity or biological influence for the envisioned application, a biosynthetic protein or peptide, a naturally occurring protein or processed naturally occurring protein, or a polysaccharide.

As noted above, thiols are of particular interest as the the nucleophilic component. Although proteins contain the amino acid cysteine, the side chain of which terminates in a thiol, there are very few free thiols within proteins. Most proteins contain an even number of cysteine residues, and these are then paired and form disulfide-cross-links between various regions of the protein. Some proteins contain an odd number of cysteine residues, and most of these are present as disulfide linked dimers, again resulting in no free thiol residues being present in the native protein. Thus, there are very few free thiols in proteins. Some important electron transferring molecules, such as glutathione, contain a free thiol, but these molecules are generally restricted in their spatial location to the inside of a cell. Conjugated unsaturated structures presented outside the cell will be substantially unreactive with most proteins at near-physiological conditions. Accordingly, using a thiol with the component containing a conjugated unsaturated bond in the mixture of the invention will react in a very self-selective manner.

In the above structures, the group P can be a polymer such as poly(ethylene glycol), poly(ethylene oxide), poly(vinyl alcohol), poly(ethylene-co-vinyl alcohol), poly(acrylic acid), poly(ethylene-co-acrylic acid), poly(ethyloxazoline), poly(vinyl pyrrolidone), poly(ethylene-co-vinyl pyrrolidone), poly(maleic acid), poly(ethylene-co-maleic acid), poly(acrylamide), or a poly(ethylene oxide)-co-poly(propylene oxide) block copolymers. This is not an exhaustive list, as other hydrophilic polymers could also be used. P can also be a copolymer, a block copolymer, a graft copolymer, or a random copolymer. Blocks, which are polymerized on the ends of the hydrophilic polymers, can be composed of, for example, lactic acid, glycolic acid, epsilon-caprolactone, lactic-co-glycolic acid oligomers, trimethylene carbonate, anhydrides, and/or amino acids, for example, to confer degradability by hydrolytic or enzymatic means.

P can also be selected to create a hydrophobic system, for example, by using a water-dispersible liquid such as polypropylene glycol. Even if P is not a hydrophobic polymer, the component containing P can be made hydrophobic, such as with pentaerythritol-tetrakis (3-mercaptopropionate) and pentaerythritol triacrylate (where the P group is pentaerythritol).

Random copolymers can be based on vinyl alcohol, such as poly(N vinylpyrroliclone-co-vinyl alcohol) or poly(ethylene-co-vinvl alcohol), with different compositions, can be derivatized with conjugated unsaturated groups, such as acrylates, 5 benzoquinones, naphthoquinones and others. The vinyl alcohol copolymers can be functionalized with (CH₂)ₙCOOH groups by reaction with ω-bromo-carboxylic acids. The resulting polymers or acrylic or methacrylic acid copolymers can be used for the attachment of quinone groups. Comonomer composition and extent of functionalization do not dramatically influence the reaction rates, unless they determine solubility or phase transition. On the other hand, they determine the final mechanical properties.

Alternatively, P may be a protein or peptide. Examples of suitable proteins and peptides for use in the invention are disclosed in International Patent Publication No. WO 00/44808, the entire disclosure of which is incorporated herein by reference.

Utilizing terminology from polymer science, polymers can be made by reaction of monomers with a functionality of 2. Cross-linked networks of polymers can be made if some or all of the monomers have a functionality greater than 2. Molecules are described herein having a functionality greater than or equal to 2 (monomers or macromers), which can be reacted together to form a cross-linked network, where functionality is defined in terms of addition reactions. As used herein, polymerization refers to the reaction of monomers or macromers with functionality of 2, and cross-linking refers to the reaction of monomers or macromers some or all of which have a functionality greater than 2. The term monomers here is not limited to small molecules, but can also refer to polymers and biopolymers.

The monomers described are of two classes, which when reacted together form a linear or cross-linked biomaterial. Both classes of monomers are required to be mixed together for cross-linking to occur. One class of monomer contains 2 or more conjugated unsaturated groups (thus, a functionality of 2 or more), preferably conjugated. The other class of monomer contains 2 or more nucleophiles (thus, a functionality of 2 or more), preferably nucleophiles that are stronger nucleophiles than others present as other components of the system.

When water-soluble or water-dispersible precursor monomers are mixed together (referred to as the final precursor solution), linear or cross-linked gels or networks are formed, and such reactions can proceed in water at physiologic or nearly-physiologic salt concentrations and pH. It is not necessary that the monomers be entirely soluble in water, and indeed it is sometimes beneficial that they not be soluble in water. In such cases, gels may not be obtained as the final material, but rather more hydrophobic, less water-swelling materials. These can be particularly useful in the delivery of hydrophobic drugs and in the formation of materials with substantial structural strength. It is only necessary that the two components be either soluble in each other or at least finely dispersible in each other, perhaps in the presence of an emulsifying agent. In this manner, the two components can come close enough to each other to react to form the linear or cross-linked material.

It is also possible to work with solutions of monomers formed in a solution other than water. For example, the use of N-methyl pyrrolidone (NMP) as a solvent in injectable biomaterial systems is known, and as such it is possible, when one wishes to work with the precursor components in solution, but with precursor components that are not freely soluble in water, to employ certain organic solvents that are acceptable for use with the sensitive biological material under consideration.

When the biomaterial is being formed in the body, as in the present invention, the list of acceptable solvents is dominated by toxicity concerns. For this application, NMP is a particularly favorable organic solvent. The toxicity of the solvent system can also be modulated by employing a mixed solvent system, comprising the organic solvent and water, to lower the overall concentration of organic solvent but to still provide good solubility or dispersability in the mixed solvent system.

Mixing to form the final precursor solution can occur in a variety of ways. Most straightforwardly, one solution contains the nucleophilic precursor component and one solution contains the conjugated unsaturated precursor component. These two components are formulated in solvent and buffer systems such that the pH and concentrations obtained after mixing are appropriate for the chemical reaction to proceed. Such mixing could occur in a static mixer at the function of two syringes, for example. Other mixing approaches can be imagined. For example, mixing can occur between fine particles of each of the two precursor solutions in an air spray.

Alternatively, one solution can be prepared from both precursor components, but at a pH, for example, such that the reaction does not proceed or proceeds only slowly. After or just immediately preceding placement of the pre-mixed precursor solution, the pH can be adjusted (e.g., by change of temperature, or mixing with acid or base, or by a chemical reaction to create an acid or base, or diffusion of an acid or base), to result in a final condition in the final precursor solution that is appropriate for the chemical reaction to proceed.

Another approach is to prepare the final precursor solution at a temperature such that the reaction does not proceed or proceeds only very slowly, either related to the activation energy of the reaction or to a buffer with temperature-sensitive characteristics or both. Upon warming or cooling (most usefully warming) to the final application temperature (e.g., to body temperature after injection), the conditions in the final precursor solution are appropriate for the chemical reaction to proceed.

Other considerations are important for the reaction occuring it situ. For example, the reactants are desirably stable in water when the precursor solutions are prepared in water. Stable is defined as reacting slowly, with slowly defined as sufficiently slow to allow the reaction between the two components to proceed and still result in the formation of the desired biomaterial. Additionally, the addition reaction in the final precursor solution is preferably not exothermic to the point of causing tissue damage, drug breakdown or other detrimental results to the biological material under consideration. The temperature of the gelling solution generally should not be raised above 60° C. during gelation, and preferably even cooler maximum reaction temperatures are desirable. Further, the components of the precursor solution must not be toxic at concentrations that diffuse out of the final precursor solution as it is applied, with the word toxic being defined as inducing a medically unacceptable tissue reaction in a medically relevant context.

In a preferred embodiment, the invention employs a thiol as the nucleophilic component and an acrylate as the component containing a conjugated unsaturated bond. Particularly preferred thiols include pentaerythritol-tetrakis (3-mercaptopropionate) (QT) and poly(ethylene glycol) hexathiol. (PEGHT). Particularly preferred acrylates include poly(ethyleneglycol)diacrylate 570 (PEGDA), poly(propylene glycol) diacrylate 900 (PPODA), pentaerythritol triacrylate (TA), and poly(ethylene glycol) tetraacrylate (QA). Acrylates react orders of mangitude faster with thiols than with amines and other mucleophiles present in biological samples, where free thiols are present in negligible leachable content. Such a system is also waterborned and, before gelations, possesses low viscosity, allowing delivery through a microcatheter. The Michael-type addition reaction, being pH-activiated, allows, for certain combinations of reagents, premixing of the reagents without reaction, while the reaction can be started at a desired time by addition of a base. The above-noted monomeric multifunctional materials are dispersed in water at high solid content, typically 75 wt %. Further, these materials can be made radiopaque by including in the reaction mixture a suitable radiopaque agent, such as barium sulfate, tantalum, iohexol (commercially available under the name Omnipaque from Amersham Health, Princeton, N.J.), iothalamate meglumine (commercially available under the name Contray from Mallinkckrodt, St. Louis, Mo.), ioxaglate meglumine and ioxaglate sodium (commercially available as a mixture under the name Hexabrix from Mallinckrodt, St. Louis, Mo.).

The liquid mixture containing the nucleophilic component and the component containing a conjugated unsaturated bond is introduced into a reproductive duct of a patient. As used herein, the term “patient” refers to human patients as well as other mammels. In female patients, the liquid mixture is introduced into one, and preferably both, of the uterine (e.g., fallopian) tubes. In male patients, the liquid mixture is preferably introduced into one, and preferably both of, the ejaculatory tracts and/or into the vas deferens.

The liquid mixture preferably also comprises a buffer, such as phosphate buffered saline (PBS). Other components can also be included within the liquid mixture, such as a base for adjusting the pH of the mixture and/or a surfactant. If a base and/or a surfactant is included in the liquid mixture, they are preferably included within a buffer solution. Exemplary bases for use in the present invention include sodium hydroxide, triethanolamine, and choline. Exemplary surfactants for use in the present invention include sorbitan monooleate, polyethylene glycol-co-polypropylene glycol, Tween 20 and Tween 80.

The mixture gels within the reproductive duct to form a gelled composition that blocks the reproductive duct. In accordance with this step of the inventive method, the Michael-type addition reaction between the nucleophilic component and the component containing a conjugated unsaturated bond is occuring predominantly, if not entirely, within the body. Accordingly, the rate of the Michael-type reaction desirably occurs over a clinically relevant period of time at a clinically relevant temperature and pH. Preferably gelation occurs over a period of less than about 60 minutes, more preferably less than about 30 minutes, still more preferably less than about 15 minutes, even more preferably less than about 5 minutes.

The speed at which the reaction occurs is largely a function of the pH of the reaction mixture, as well as the strength of the buffer solution employed. For example, a liquid mixture containing pentaerythritol-tetrakis (3-mercaptopropionate) (QT) and poly(ethyleneglycol)-diacrylate 570 (PEGDA) in 100 mM PBS solution at pH 7.4 reacts to form a gel in about 5 minutes. In contrast, a liquid mixture containing QT and PEGDA in 10 mM PBS solution will react in about 10 minutes if adjusted to a pH of about 9. The strength of the buffer solution is preferably sufficient to deprotanate the thiols in the liquid mixture. Preferably the liquid mixture contains a PBS solution having a strength ranging from about 1 mM to about 300 mM, more preferably from about 10 mM to about 150 mM, still more preferably from about 75 mM to about 125 mM. Preferably the pH of the liquid mixture, when it is being introduced into the body, is at least 7, more preferably from about 7 to about 12. If the liquid mixture has a pH outside of this range, the pH can be adjusted immediately before introduction into the patient by addition of a suitable base, as noted above.

The liquid mixture can be introduced into the reproductive duct of the patient by any suitable method. In a female patient, the liquid mixture is preferably introduced transvaginally by a catheter using a visually guided technique, hysteroscopy. For such a procedure, the catheter preferably has a size no greater than about 5 French so that it fits within the hysteroscope. A suitable catheter for use in connection with the invention is commercially available from Conceptus, Inc. (San Carlos, Calif.) in connection with the Essure™ contraception system. Other catheters could also be used to introduce the liquid mixture. A catheter would desirable include a backstop mechanism, such as a balloon, to prevent backflow of the liquid mixture into the uterus prior to gelling. In male patients, a catheter can similarly be used for introduction of the liquid mixture.

If desired, the liquid mixture can further include one or more additional agents, such as a progestin (including progesterone and all other 18-, 19- and 21-carbon human steroids and steroid precursors), a spermicidal compound, a nonsteroidal anti-inflammatory compound, an anti-prostaglandin compound, a quinacrine or another agents that causes mucosal and/or tubal scarification. In particular, the presence of progesterone in the gelled product in the fallopian tube can increase the efficacy of the biomaterial.

### EXAMPLES

For the following examples, unless otherwise indicated, pentaerythritol-tetrakis (3-mercaptopropionate) (QT) and pentaerythritol triacrylate (TA) were both obtained from Fluka (Buchs, Switzerland). Poly(ethylene glycol) diacrylate 570 MW (PEGDA), poly(propylene glycol) diacrylate 900 MW (PPODA 900), and poly(propylene glycol-co-polyethylene glycol-co-polypropylene glycol) 3300 MW (PEP) were all obtained from Aldrich (Buchs, Switzerland). Sorbitan monooleate (SM) was purchased from Sigma (Buchs, Switzerland). All other reagents were reagent grade and all materials were used as received.

### Example 1

### Synthesis of poly(ethylene glycol) hexathiol (PEGHT)

The QT (8.58 g, 17.5 mmol) was combined with 8 mL H₂O and 2 mL 1N NaOH in 100 mL of tetrahydrofuran (THF). The PEGDA 570 (1 g, 1.75 mmol) was combined with 1 mg of 2,6-di-tertbutyl-p-chresol (radical inhibitor) in 10 mL di-chloromethane (DCM). The PEGDA solution was then diluted with 15 mL of THF. The diluted PEG solution was then dropwise added to the stirred QT solution. After 55 min the pH was adjusted to 7 using glacial acetic acid. To remove water, the solvent was evaporated and the product was redissolved in 100 mL of toluene. The toluene was evaporated and an additional 100 mL of toluene was added. About 13 g of sodium sulfate was added, and then the sodium sulfate was removed by filtering. Before precipitation in 10-fold excess diethyl ether, the solution was concentrated by evaporating some of the toluene. The diethyl ether was decanted and the liquid product was recovered. The product was dried under vacuum.

¹H-NMR (CDCl₃): δ=4.25 (t, 2H, —OCH₂CH₂OOC—), 3.6-3.7 (40+16H, CH₂CH₂O and C(═O)OCH₂C(CH₂—)₃), 2.8 (m,8H, SCH₂CH₂COO), 2.65 (m, 8H,SCH₂CH₂COO), 1.65 (t, 3H, -CH₂SH) ppm.

Gel permeation chromatography (GPC): Mₙ=1300; M_{w}=1500 (THF, PEG standards).

### Example 2

### Synthesis of poly(ethylene glycol) tetraacrylate (PEGOA)

The PEGDA 570 (20 g, 35 mmol) was combined with 20 mg of 2,6-di-tertbutyl-p-chresol in 10 mL DCM. This was then diluted with 90 mL THF. The NaOH (3 mL, 0.2N) and 1 mL of H₂O were added. The QT (1 g, 2.0 mmol) was dissolved in 40 mL of THF. The QT solution was added dropwise to the stirred PEGDA solution. After 30 min, 7 μL of glacial acetic acid was added to neutralize the reaction. The solvent was evaporated and 100 mL of toluene were added. After drying over sodium sulfate, the solution was filtered, concentrated and then precipitated using 10-fold excess diethyl ether. After precipitation the product was dried under vacuum.

¹H-NMR (CDCl₃): δ=6.4 (dd, 4H, CH₂=CHCOO), 6.15 (dd, 4H, CH₂=CHCOO trans), 5.85 (dd, 4H, CH₂=CHCOO cis), 4.25 (t, 2H -OCH₂CH₂OOC—), 3.6-3.7 (40+16H, CH₂CH₂O and C(═O)OCH₂C(CH₂—)₃), 2.8 (m, 8H, SCH₂CH₂COO), 2.65 (m, 8H, SCH₂CH₂COO) ppm.

GPC: Mₙ=2600; M_{w}=2900 (THF, PEG standards)

### Example 3

### Preparation of Crosslinked Biomaterials

Crosslinked materials were prepared as dispersions or reverse emulsions of precursors in modified phosphate buffered saline (PBS). The PBS, 10 mM solution, was obtained by mixing equal volumes of 10 mM PBS adjusted to pH 9 with the addition of triethanolamine or 1N NaOH, respectively.

### Example 3A

As a typical procedure for 75-wt % dispersion materials, 424 mg QT and 997 mg of PEGDA 570 were combined and mixed well by vortexing. Air bubbles were removed by sonication. The PBS solution (473 mg) was added to the mixed precursors. The mixture was again vortexed for about 2 min to mix well and disperse the precursors in the aqueous solution. Following vortexing, the mixture was again sonicated to remove air bubbles. The materials were then allowed to gel at either 25° C. or 37° C.

### Example 3B

Dispersion-type materials with barium sulfate inorganic particles (as a radiocontrast agent) were prepared according to the protocol described in Example 3A. Before addition of the activating buffer (pH 9.0 PBS), 10 wt % of 0.8 μm BaSO₄ particles were added to the mixed precursors. The activating buffer was then added, and the mixture was vortexed and allowed to crosslink.

### Example 3C

SM was added to the PBS 9.0 buffer at 4 wt % before adding the mixed precursors, QT and PEGDA 570, in amounts as described in Example 3A.

### Example 3D

Dispersion-type materials at 75 wt % solid were prepared as described in Example 3C using the prereacted precursors, PEGHT and PEGQA at a 1:1 thiol to acrylate ratio.

### Example 3E

A reverse-emulsion material was prepared. 1223 mg QT (2.5 mmol) was mixed with 993 mg TA (3.3 mmol) and 90 mg PEP. The PBS buffer, with 0.1N NaOH, (738 mg) was added, and the mixture was vortexed thoroughly. The mixture was then allowed to crosslink;

Crosslinking kinetics—shear rheology

The dispersions or reverse emulsions at 75 wt % content were placed between two parallel plates (separated by 100 μm) of a CVO120 Rheometer (Bohlin Instruments) at 25° C. A constant oscillatory strain of 3×10⁻¹ at 1 Hz was applied. The gel point was defined as the time when the phase angle was equal to 45° (i.e., loss modulus equal to the storage modulus). The mean gel time and mean rate of change in the phase angle at gelation for five samples were determined for each of these three materials.

Cross-linking kinetics—attenuated total reflection-infrared spectroscopy (ATR-IR)

The QT and PEGDA (75 wt %) dispersion gels were prepared with 1:1 thiol to acrylate ratios and 2:1 ratios to demonstrate the 1:1 consumption of thiols and acrylates by quantifying the S—H stretching vibration at 2600 cm⁻¹ and the olefin out-of-plane C—H bending vibration at 800 cm⁻¹. Both peaks were normalized in intensity, referring to the peak of C—H stretching vibrations, the intensity of which should not change dramatically during the reaction.

Morphology

The physical morphology of 75 wt % QT/PEGDA dispersions and QT/PPODA reverse emulsions was investigated using scanning electron microscopy (SEM) on a JEOL 255 electron microscope. Dispersion materials (75 wt % solid) were also imaged using light microscopy at 100× in bright field.

Mechanical Properties

Mechanical properties in compression were investigated for dispersion-type materials with and without added surfactant or radiopaque inorganic particles, for dispersion-type materials using prereacted precursors, and for reverse emulsion-type materials. The effect of the thiol-to-acrylate ratio on material ultimate strength and ultimate deformation was also investigated in the dispersion materials by adjusting the relative quantities of QT and PEGDA 570, keeping the solid content at 75 wt %.

Mechanical properties were evaluated for the reverse emulsion-type materials, varying the solid content and the crosslinking density (by substituting TA and PPODA 900).

Each gelling mixture was poured into polyethylene tubes with 8 mm i.d. and allowed to cure for 24 h. Following gelation, the material was cut into 6-mm-long sections, using a metal frame to standardize the sample size and ensure sectioning perpendicular to the axis of the cylindrical sample. After 24 h in 10 mM PBS, the gels were compressed with a Zwick Z005/TN2S mechanical tester (Zwick GmbH, Ulm, Germany) with a 5 kN load cell at 10 mm/min to gel σₘₐₓ (ultimate strength) and δₘₐₓ (ultimate deformation) data. At least three samples for each material type were tested.

The addition of SM decreased the gel point from 9.4±0.6 min. to 6.4±0.4 min (n=3, p=0.001). The reverse-emulsion material showed an even shorter gel point, 4.9±0.8 min, but with a more gradual transition: d(phase angle)/dt of only 12±3°/min compared with 57±4°/min (for dispersion-type materials without SM) or 83±6°/min (dispersion-type materials with SM).

Monitoring the depletion of thiols and acrylates as a function of time showed that both with 1:1 and 2:1 thiol-to-acrylate formulations the two groups followed similar depletion kinetics. Additionally, at 1:1 ratio, no unreacted groups were visible at the end of the reaction, whereas in the 2:1 case the thiols were reduced to half their original concentration, whereas the acrylates were reduced to nearly zero at the end of the reaction.

The SEM images showed the course and likely interpenetrated structure of the dispersion materials, and the continuous organic phase of the reverse-emulsion ones.

From compression stress-strain curves, the ultimate strengths and ultimate deformations were determined at 75 wt % solid content. For the dispersion material, ultimate strength of 1.8±0.2 MPa and ultimate deformation of 35 +2% were observed. An increase in the ultimate deformation was noted upon the use of an emulsifier, probably because of the higher homogeneity of the sample; apparently the use of prereacted precursors or the addition of the inorganic radiopaque material did not improve the stability of the dispersion as much as the emulsifier. In the corresponding reverse emulsion-type material the continuous organic phase gave a much stronger material (6.7±0.5 MPa ultimate strength) while demonstrating a similar ultimate deformation (37±2%).

For reverse-emulsion materials the ultimate strength, ultimate deformation, and Young's modulus increased with increased solid content or increased crosslinking density seen by exchanging a higher-molecular-weight precursor, PPODA 900 with the low-molecular-weight TA.

### Example 4

For in situ gelation, 424 mg QT and 997 mg PEDGA 570 (both sterilized by sterile filtration (0.2 um filter)) are loaded into sterile syringes. The QT and PEGDA are mixed by static mixing (forcing the two components back and forth together, between the two syringes through a sterile female to female syringe adaptor). After the two components are well mixed, the contents are loaded into one syringe. As an activator, a PBS, 10 mM solution, is obtained by mixing equal volumes of 10 mM PBS adjusted to pH =9 with the addition of triethanolamine or 1N NaOH. The activator, sterilized by sterile filtration, is loaded into another sterile syringe. The activator is combined with the two mixed precursors by static mixing for 2 minutes. Thereafter, the activated material is introduced through a catheter using the syringe into a fallopian tube of a patient. With this mixture, there is a time period of about 8 mintues to deliver the material through the catheter and into the fallopian tube after mixing the activator. This time can be increased by lowering the pH of the activator. Anesthsia for the transcervical procedure is accomplished with intrmuscular injections of 20 mg/kg xylazine hydrochloride and 50 mg/kg ketamine hydrochloride.

### Example 5

A rabbit was sedated with Telazol (72 mg) & Xylazine (24 mg) IM followed by 1.5% isoflurane in 1 L oxygen. After sedation, the rabbit's abdominal area was shaved with a #40 blade and prepped with Betadine scrub, alternating removal of Betadine with alcohol soaked gauze three times and a final application of Betadine solution was left on the skin. The rabbit was placed on the operating room table in dorsal recumbency position. A single pre-operative dose of Ancef (120 mg IM) was given. Monitoring leads were placed and general anesthesia was administered. The rabbit was draped. A 5 cm low-vertical incision was made with the scalpel and carried down to the underlying fascial structures. The peritoneal cavity was entered sharply with attention to underlying structures. The incision was extended superiorly and inferiorly with Mayo scissors. The bladder was immediately observed protruding from the incision. Gentle manual compression expressed the urine allowing for better visualization.

The didelphys uterus was exteriorized. Attention was turned to the right uterine body. A hysterotomy was created one centimeter from the palpable tubal ostia with an 18G needle. The 18G was removed and a blunt-ended 21G needle introduced through the uterine incision and into the ostia. A moist sponge was placed over the uterus and a long mayo clamp was used to occlude the portion of the uterine body medial to the cannulating needle. Chromopertubation verified patency. A liquid composition comprising 1.112 g PEGDA and 0.365 g QT in a buffer solution containing 0.490 g PBS (100 mM/pH 7.4) was then introduced with noticeable spillage from the fimbria. Chromopertubation was then performed on the left uterine tube (control) without occlusion of the uterine body utilizing the same procedure.

The uterus was then returned to the abdomen. The fascia was closed with a running suture of 0-vicryl on a CT-1 needle. The skin was closed with subcuticular 4-0 monocryl on a PS-1 needle. Dermabond skin glue was applied to the incision site. The rabbit tolerated the operative procedure well and awoke from anesthesia without difficulty. A second dose of Ancef was given post-operatively.

### Example 6

For this example, pentaerythritol-tetrakis (3-mercaptoproprionate) (QT), poly(ethylene glycol) diacrylate (PEGDA) having an average molecular weight of 575, poly(propylene glycol) diacrylate (PPODA) having an average molecular weight of 540, sodium chloride, sodium hydroxide (1.005 N, volumetric standard), and progesterone were obtained from Aldrich company, Milwaukee, USA and used as received. Sodium phosphate (dibasic), sodium phosphate (monobasic), and methanol were from Sigma Chemical Company, St. Louis, USA.

Progesterone loaded cylinders (1.6 mm×1.0 cm) were prepared using systems containing QT with either PEGDA or PPODA. For the PEGDA samples, 33 mg (5.5-wt %) or 165 mg (27-wt %) of progesterone (in powder form) was first weighed into a 1.8 ml cryovial. 288 mg of PEGDA was then added, and the PEGDA and progesterone were mixed by vortexing the vial. Next, 122 mg of pentaerythritol-tetrakis (3-mercaptoproprionate) (QT) was added, and the resulting mixture was vortexed for 90 sec to premix. Following the last vortex, 136.7 mg of 0.1 M PBS (pH 7.35) was added, and the solution was vortexed for 2 min to ensure thorough mixing. Following this, the mixture was injected into a 6 to 8 cm long tygon tube having an inner diameter of 1.6 mm. The ends of the tube were sealed with parafilm, and the gelling mixture was allowed to cure overnight. The tubing was then cut away, and the intact gel was cut into 1 cm long samples.

For the PPODA samples, 32 mg (5.8-wt %) or 160 mg (23-wt %) of progesterone was weighed into a 1.8 cm cryovial. Next, 270 mg of PPODA was added and mixed. Following this,122 mg of QT was added, and the entire mixture was premixed by vortex. Next, 130.7 mg of 0.1 M PBS (pH 12 adjusted with 1 N NaOH) was added, and the mixture was vortexed for 2 min. The mixture was injected into a tygon tube, cured, and cut into 1 cm long samples.

Weighing the samples and calculating the fraction of the volume in each sample compared to the total mixture volumes approximated drug loading. The drug loading was confirmed by UV spectroscopy at 247 nm after the release experiments by extracting the remaining progesterone in 10 mL of methanol for 2 weeks (ε=0.0185 (mass extinction coefficient for progesterone in methanol, R²=0.99) mL/(μg cm)).

All the drug-loaded samples (3 each for PEGDA and PPODA at each drug loading) were placed individually into labeled 50 ml centrifuge tubes. PBS (50 ml, pH 7.35, 0.1 M) was added to each tube. The tubes were capped and placed inside a rocker-incubator set at 37° C., 75 rpm. In order to measure progesterone release, 1 ml aliquots were taken from each centrifuge tube after 24 hrs. The concentration of the drug in solution was determined by UV absorbance at 247 nm (ε=0.0341 (mass extinction coefficient for progesterone in PBS), mL/(μg cm)). The remaining PBS in each centrifuge tube was discarded and replaced with 50 ml of fresh PBS. Sink conditions were maintained by replacing the PBS at sufficient intervals to keep the concentration below 20% of the saturation limit in the buffer (every 24-72 hours, on average).

To investigate whether these injectable delivery systems could be used for long-term progesterone release within the fallopian tubes and to evaluate for zero-order release, the release profiles were analyzed to determine dependence of the release on drug loading and time. The profiles were analyzed using Equation 1:

$$\frac{{M}_{t}}{{M}_{∞}} ≡ k \cdot {t}^{n}$$

where Mₜ/M_{∞} is the fraction of total progesterone released at time t, t is the release time, and k is a kinetic constant dependent on the system geometry and diffusion coefficients. The n-values from Equation 1 were calculated for the progesterone release profiles by linear regression using least squares estimates.

The swelling of these materials was evaluated gravimetrically. PEGDA and PPODA samples were prepared as outlined in the sample preparation section but without progesterone and with a diameter of 2.4 mm. The cut samples (˜1 cm in length) were then placed in PBS (0.002 M) at pH 7.4 and weighed at regular intervals. Lengths and diameters were measured using calipers. Following equilibrium, the samples were dehydrated completely under vacuum at 70° C. The swelling ratio, q, was calculated as q=(w_{w}−w_{d})/w_{w}: where w_{w} is the wet weight and w_{d} is the dry weight.

PEGDA and PPODA samples were prepared as described in the sample preparation section. The cut surfaces of the PEGDA and PPODA samples were imaged using Scanning Electron microscopy (1000× magnification on a JOEL 840 Scanning electron microscope).

Progesterone was released from new waterborne in situ-gelling, self-reactive materials. The release profile was analyzed using Equation 1.

The above-described systems possessed multiphase release, similar to that found in early hydrophobic membrane systems. The first phase involved some burst effect of surface drug. The second phase, most importantly, was partition-controlled, zero-order release. The final phase was the diffusion controlled release attributed to drug depletion. It was found that changes in the drug loading, about 5 wt % to about 25 wt %, did not change the steady state release rate from these systems. There was a marginal increase in the steady state release rate in the PEGDA systems compared to the PPODA systems, despite the significant difference in PPODA and PEGDA hydrophobicity. This increase is accounted for by the increased surface area of the PEGDA sample compared to the PPODA sample due to the difference in swelling.

In the PEGDA system, the two loading levels showed similar release profiles for the first 200-500 hours. At later times (beyond 500 hours), the release rate decreases in the lower drug loading samples relative to the higher loaded samples. This is due to the depletion of dispersed drug and the concentration, below saturation, is no longer constant. As in the PEGDA system, the two loading levels exhibited similar release profiles in the PPODA system for the first approximately 250 hours. Similar to PEGDA, the PPODA system with the lower drug loading showed a decrease in the mass released rate after about 400 hours. In both cases the actual mass release rate is not dependent on loaded concentration.

The diffusional release exponent values, greater than 0.5, indicate that there is some anomalous release mechanism. This occurs because the overall release profile is a combination of different release mechanism including both first and zero-order release at different times. For the PEGDA samples, the exponents for the steady state phase suggest a zero-order release mechanism during phase ‘B’. The release is constant with time during this phase. The release rates between high and low drug loadings were not statistically different in either case. However, the release was significantly lower in the PPODA system compared to PEGDA system. This is partially attributed to the difference in the surface area of the two systems due to the difference in swelling. It may also be partially dependent on the higher hydrophobicity of the PPODA compared to PEGDA and the lipophilic nature of the progesterone.

The preceding description has been presented with reference to presently preferred embodiments of the invention. Workers skilled in the art and technology to which this invention pertains will appreciate that alterations and changes in the described methods may be practiced without meaningfully departing from the principal, spirit and scope of this invention. Accordingly, the foregoing description should not be read as pertaining only to the precise methods described, but rather should be read consistent with and as support to the following claims which are to have their fullest and fair scope.

1. A method for sterilizing a patient comprising introducing into a reproductive duct of the patient a composition comprising a nucleophilic component and a component containing a conjugated unsaturated bond, whereby the composition undergoes crosslinking within the reproductive duct.

2. The method according to claim 1, wherein the nucleophilic component is selected from the group consisting of thiols, amines and mixtures thereof.

3. The method according to claim 1, wherein the nucleophilic component is a thiol.

4. The method according to claim 1, wherein the nucleophilic component is selected from the group consisting of pentaerythritol-tetrakis(3-mercaptopropionate) and poly(ethylene glycol) hexathiol.

5. The method according to claim 1, wherein component containing a conjugated unsaturated bond is selected from the group consisting of acrylates, vinylsulfones, acrylamides, quinones and vinylpyridiniums.

6. The method according to claim 1, wherein the component containing a conjugated unsaturated bond is an acrylate.

7. The method according to claim 1, wherein the component containing a conjugated unsaturated bond is selected from the group consisting of poly(ethyleneglycol)diacrylate, poly(propylene glycol) diacrylate, pentaerythritol triacrylate, and poly(ethylene glycol) tetraacrylate.

8. The method according to claim 1, wherein the nucleophilic component is a thiol and the component containing a conjugated unsaturated bond is an acrylate.

9. The method according to claim 8, wherein the nucleophilic component is selected from the group consisting of pentaerythritol-tetrakis(3-mercaptopropionate) and poly(ethylene glycol) hexathiol.

10. The method according to claim 9, wherein the component containing a conjugated unsaturated bond is selected from the group consisting of poly(ethyleneglycol)diacrylate, poly(propylene glycol) diacrylate, pentaerythritol triacrylate, and poly(ethylene glycol) tetraacrylate.

11. The method according to claim 8, wherein the component, containing a conjugated unsaturated bond is selected from the group consisting of poly(ethyleneglycol)diacrylate, poly(propylene glycol) diacrylate, pentaerythritol triacrylate, and poly(ethylene glycol) tetraacrylate.

12. The method according to claim 1, wherein the composition further comprises a buffer solution.

13. The method according to claim 1, wherein the composition further comprises a surfactant.

14. The method according to claim 1, wherein the liquid further comprises a base.

15. The method according to claim 1, further comprising increasing the pH of the composition prior to introducing the composition into the reproductive duct.

16. The method according to claim 1, wherein the composition gels within the reproductive duct within 30 minutes of introduction.

17. The method according to claim 1, wherein the composition gels within the reproductive duct within 15 minutes of introduction.

18. The method according to claim 1, wherein the composition is introduced into the reproductive duct through a catheter.

19. The method according to claim 1, wherein the composition further comprises at least one additional agent selected from the group consisting of radiopaque agents, progestins, spermicidal compounds, nonsteroidal anti-inflammatory compounds, anti-prostaglandin compounds, quinacrine, and agents that cause mucosal and/or tubal scarification.

20. The method according to claim 1, wherein the composition further comprises progesterone.

21. The method according to claim 1, wherein the composition further comprises a radiopaque agent.

22. The method according to claim 1, wherein the composition comprises:
(a) a buffer solution;
(b) a nucleophilic component selected from the group consisting of pentaerythritol-tetrakis(3-mercaptopropionate) and poly(ethylene glycol) hexathiol;
(c) a component containing a conjugated unsaturated bond selected from the group consisting of poly(ethyleneglycol)diacrylate, poly(propylene glycol) diacrylate, pentaerythritol triacrylate, and poly(ethylene glycol) tetraacrylate; and
(d) a surfactant.

23. The method according to claim 22, wherein the surfactant is selected from the group consisting of sorbitan monooleate and polyethylene glycol-co-polypropylene glycol.

24. A method for sterilizing a female patient comprising introducing into at least one uterine tube of the patient a composition comprising a nucleophilic component and a component containing a conjugated unsaturated bond, whereby the composition crosslinks within the at least one uterine tube.

25. A method for sterilizing a male patient comprising introducing into at least one ejaculatory duct and/or the vas deferens of the patient a composition comprising a nucleophilic component and a component containing a conjugated unsaturated bond, whereby the composition cross-links within the at least one ejaculatory duct and/or the vas deferens.
